SEQUENCE LISTING (9 sequences)

SEQ ID NO: 1 - DNA, 24 bp, Artificial
  Synthetic primer
cccaagatggcggccaccatgaag

SEQ ID NO: 2 - DNA, 24 bp, Artificial
  Artificial primer
gtttaactttcatcagcttcaagg

SEQ ID NO: 3 - DNA, 26 bp, Artificial
  Artificial primer
tgaaggtcggagtcaacggatttggt

SEQ ID NO: 4 - DNA, 24 bp, Artificial
  Artificial primer
catgtgggccatgaggtccaccac

SEQ ID NO: 5 - protein, 14 aa, Artificial
  Artificial peptide
AKFGSNINLEADES

SEQ ID NO: 6 - DNA, 21 bp, Artificial
  Artificial primer
uucagcgaguguuagcagatt

SEQ ID NO: 7 - DNA, 20 bp, Artificial
  Artificial primer
ucugcuaacaccgcugaatt

SEQ ID NO: 8 - DNA, 1345 bp, Homo sapiens
aaagtccaagaggacggaatgtggagacagtgttgtatttttgcggggagttctaggccgaccgggagcgagagaacgctcgggggcgaagcgcgccattgcggccctccccgccgcctgcggtagtccagtcccaagatggcggccaccatgaagaaggcggctgcagaagatgtcaatgttactttcgaagatcaacaaaagataaacaaatttgcacggaatacaagtagaatcacagagctgaaggaagaaatagaagtaaaaaagaaacaactccaaaacctagaagatgcttgtgatgacatcatgcttgcagatgatgattgcttaatgataccttatcaaattggtgatgtcttcattagccattctcaagaagaaacgcaagaaatgttagaagaagcaaagaaaaatttgcaagaagaaattgacgccttagaatccagagtggaatcaattcagcgagtgttagcagatttgaaagttcagttgtatgcaaaattcgggagcaacataaaccttgaagctgatgaaagttaaacattttataatactttttttatttgtttaataaacttgaatattgtttaaaatgataattttccttcttcaaatgacatggaaagcaaaactttcttttttaaaaattttcatttatttaatggaaacttgcccattttcacatgtctgcttatttattttatatttttaaaagaagacagtattcacctatgtattttgcataacgattatatcaagtctaggggcttcatgtcatgttattaaaatcagttaagcaatcttttatgtttctatattatttagaatatttgttgttgcaattttcacataagaaaatttaacagttgtgtcatgttgtttctgtctgattttaattgctgtctaatgacggggaaagcacgatgaaaagatgtacaatcctgcatccttgcttatttcacaactaaagctttgtcatagacttcaaaatatatatgtatatattttatttaaatatatgttacatattatatttaaacatacatatttaacattttttacatatctatcaatatcagagatttgggtaaaagaatgggtaatgtttaaacatgtggaggcatgtggagctttatacaaacagggcagaaccacagaagaacgttttagaaaccaagagatgtgcagaaagaaatgtttagtgttttttcgttttaaattttagattttattttagtgctttgtaattaattggggtttatattgataaagatgtggaagttaaacagctatgtatgtaaaagtaaggcttatttcttaaataaaggatgcatttcttccc

SEQ ID NO: 9 - protein, 134 aa, Homo sapiens
MAATMKKAAAEDVNVTFEDQQKINKFARNTSRITELKEEIEVKKKQLQNLEDACDDIMLADDDCLMIPYQIGDVFISHSQEETQEMLEEAKKNLQEEIDALESRVESIQRVLADLKVQLYAKFGSNINLEADES

(19) United States
(12) Patent Application Publication
(10) Pub. No.: US 2012/0070486 A1
(43) Pub. Date: Mar. 22, 2012

(54) METHODS AND COMPOSITIONS RELATED TO PREFOLDIN AND ITS REGULATION

(76) Inventors: Anil K. Sood, Pearland, TX (US); Rosemarie Schmandt, Houston, TX (US); Gabriel Lopez-Berestein, Bellaire, TX (US)

(21) Appl. No.: 12/526,756

(22) PCT Filed: Feb. 12, 2008
(86) PCT No.: PCT/US2008/053702

§ 371 Date: Jan. 20, 2010

Related U.S. Application Data
(60) Provisional application No. 60/889,535, filed on Feb. 12, 2007.

(51) Int. Cl. A61K 9/127; A61K 31/7088; A61K 9/00; A61P 35/00; A61K 38/12; A61K 31/704; A61K 38/14; C07H 21/00; C07H 21/02; A61K 33/24

(52) U.S. Cl. 424/450; 536/24.5; 514/44 A; 424/400; 424/649; 514/19.9; 514/34; 514/19.3

(57) ABSTRACT

The present invention relates to the fields of molecular biology and drug delivery. In certain embodiments, the present invention provides methods for the delivery of a siNA (e.g., a siRNA) to a cell to modulate expression of a PFDN 1-6. These methods may be used to treat a disease, such as cancer.

The present invention claims priority to U.S. Provisional Patent Application Ser. No. 60/889,535, filed Feb. 12, 2007, which is incorporated by reference herein in its entirety.

The present invention was supported by The University of Texas MD Anderson OVARIAN SPORE grant number IP50CA83639.

### BACKGROUND OF THE INVENTION

I. Field of the Invention

The present invention relates generally to the fields of molecular biology, medicine, oncology, and delivery of therapeutic compounds. More particularly, it concerns the delivery of inhibitory nucleic acids that inhibit the function, activity, or expression of PFDN4 gene or transcript, including siNA (e.g., a siRNA) to a hyperproliferative or a cancer cell.

II. Description of Related Art

The major components of the eukaryotic cytoskeleton include microtubules, actin microfilaments, and intermediate filaments. The importance of these intracellular components, and their utility as therapeutic targets for the treatment of cancer, has long been recognized. In particular, drugs that target microtubules including taxanes, epithiliones, and vinca alkaloids are among the most commonly prescribed chemotherapeutic agents for the treatment of a variety of solid tumors (Zhou and Giannakakou, 2005; Pellegrini and Budman, 2005; Jordan and Wilson, 2004).

The taxanes are diterpenes produced by the plants of the genus Taxus (yews). As their name suggests, they were first derived from natural sources, but some have been synthesized artificially. Taxanes include paclitaxel and docetaxel. Paclitaxel was originally derived from the Pacific yew tree.

Taxanes have been used to produce various chemotherapy drugs. The principal mechanism of the taxane class of drugs is the disruption of microtubule function. It does this by stabilizing GDP-bound tubulin in the microtubule. Microtubules are essential to cell division, and taxanes therefore stop this—a “frozen mitosis”. Thus, taxanes are essentially mitotic inhibitors. In contrast to the taxanes, the vinca alkaloids destroy mitotic spindles. Both, taxanes and vinca alkaloids are therefore named spindle poisons or mitosis poisons, but they act in different ways. Taxanes are also thought to be radiosensitizing.

Both paclitaxel and docetaxel, which are routinely used in the treatment of ovarian cancer (Berkenblit and Cannistra, 2005; Cannistra, 2004; Ozols et al., 2004), bind directly to the tubulin subunits of the microtubules and stabilize these otherwise dynamic structures. By interfering with normal microtubule dynamics, these drugs impact intracellular transport, cell signaling, cellular structure and locomotion, and disrupt mitotic spindle formation, resulting in the death of rapidly dividing cancer cells. Unfortunately, acquired chemoresistance is all too common in recurrent ovarian cancer, and patients eventually succumb to their disease. Mechanisms contributing to taxane resistance include differential expression of tubulin isotypes and mutations in tubulin that negatively affect taxane binding, in addition to the over-expression of multidrug resistance genes (p-glycoprotein, MRP) which actively export these drugs from chemoresistant cells (Zhou and Giannakakou, 2005; Pellegrini and Budman, 2005; Jordan and Wilson, 2004; Berkenblit and Cannistra, 2005; Cannistra, 2004; Ozols et al., 2004; Baird and Kaye, 2003).

### SUMMARY OF THE INVENTION

To address the above needs for cancer therapeutics, the inventors target the cytoskeleton of hyperproliferative and/or cancerous cells, in particular therapy resistant cells, using novel strategies to circumvent resistance to various chemotherapies. Certain embodiments of the invention include targeting cellular components required for cytoskeletal assembly as opposed to targeting the microtubule cytoskeleton directly. In certain aspects, the chaperone, prefoldin (PFDN/GimC), and specifically the PFDN4 subunit, has been identified as a druggable target.

Embodiments of the invention include methods of treating a cancer cell comprising administering to a cancer an amount of an inhibitor of Prefoldin activity, transcription, and/or translation.

The present invention provides compositions and methods for delivery of an inhibitory nucleic acid, including short interfering ribonucleic acids (siRNA) or nucleic acids that encode siRNAs. In certain embodiments the inhibitory nucleic acid can be delivered to a cell using liposome delivery vehicle. In certain aspects, the liposome delivery vehicle is a non-charged (neutral) liposome. Liposomes may be used to efficiently deliver an inhibitory nucleic acid such as a siNA or a siRNA to cells in vivo. In further aspects, methods of the present invention may be particularly suited for the treatment of cancer or other hyperplastic conditions. Methods of the invention can be used to augment a therapeutic effect, sensitize a cancer cell to other traditional therapies or anticancer drugs, or be used as a therapeutic composition alone or in combination with other anticancer therapies.

Embodiments of the present invention relate to compositions comprising a siNA component, particularly an siNA that targets a PFDN encoding nucleic acid, in particular a PFDN4 encoding nucleic acid. In certain aspects, the PFDN siNA or other PFDN inhibitor can be complexed with one or more a lipid component. A lipid component can comprise one or more phospholipids. The lipid component may have essentially a neutral, a positive, or a negative charge. In certain aspects the lipid component may be in the form of a liposome. The siNA (e.g., a siRNA) may be encapsulated in the liposome or lipid component, but need not be. Encapsulate refers to the lipid or liposome forming an impediment to free diffusion into solution by an association with or around an agent of interest, e.g., a liposome may encapsulate an agent within a lipid layer or within an aqueous compartment inside or between lipid layers. In certain embodiments, the composition is comprised in a pharmaceutically acceptable carrier. The pharmaceutically acceptable carrier may be formulated for administration to a human subject or patient.

An inhibitory nucleic acid (siNA) includes a siRNA (short interfering RNA) or shRNA (short hairpin RNA), a dsRNA (double stranded RNA), a ribozyme, an antisense nucleic acid molecule or a nucleic acid encoding thereof that specifically hybridize to a nucleic acid molecule encoding a target protein or inhibiting the expression of the target protein. “Specific hybridization” means that the siRNA, shRNA, ribozyme or antisense nucleic acid molecule hybridizes to the targeted nucleic acid molecule and inhibits its expression. Preferably, “specific hybridization” also means that other genes or transcripts are not affected or substantially affected. A siNA can be a double-stranded nucleic acid and may comprise 18, 19, 20, 21, 22, 23, 24, 25, 26, 27, 28, 29, 30, 35, 40, 50, 100 to 18, 20, 21, 22, 23, 24, 25, 26, 27, 28, 29, 30, 40, 50, 100, 200, 300, 500 or more nucleobases or nucleobase pairs, in particular a segment of a PFDN gene or nucleic acid encoding a PFDN protein, including 5′ and 3′ non coding regions of a nucleic acid encoding a PFDN protein. In particular aspects the double stranded nucleic acid can comprise 18 to 30, 19 to 25, 20 to 23, or 21 contiguous nucleobases or nucleobase pairs. In certain embodiments, the siNA inhibits the translation of a gene that promotes growth of a cancerous or pre-cancerous or hyperplastic mammalian cell (e.g., a human cell), in particular a gene or gene product, e.g., an mRNA etc., that encodes a protein that assists in producing stable cytoskeletal elements. An siNA may induce apoptosis in the cell, sensitize a cell to other antigrowth agents, and/or inhibit the transcription, transport, processing, and/or translation of a mRNA or other target gene. In certain embodiments, the siNA component comprises a single species of siRNA. In other embodiments, the siNA component comprises a 2, 3, 4 or more species of siRNA that target 1, 2, 3, 4, or more genes, particularly one or more PFDN gene or transcript. Compositions of the invention may further comprise a chemotherapeutic or other anti-cancer agent, which may or may not be encapsulated in a lipid component or liposome. In further embodiments, the nucleic acid component is encapsulated within the liposome or lipid component.

Another aspect of the present invention involves methods for delivering siNA to a cell comprising contacting the cell with a lipid composition. The methods typically provide an inventive composition in an effective amount. An effective amount is an amount of therapeutic component that modifies, enhances, or augments the effect of a drug or therapy; sensitizes a cell to a second therapy; or attenuates, slows, reduces or eliminates a cell, condition or disease state in a subject. The cell may be comprised in a subject or patient, such as a human. The method may further comprise a method of treating cancer or other hyperplastic condition. The cancer may have originated in the ovary, bladder, blood, bone, bone marrow, brain, breast, colon, esophagus, gastrointestine, gum, head, kidney, liver, lung, nasopharynx, neck, prostate, skin, stomach, testis, tongue, or uterus. In certain embodiments, the cancer is ovarian cancer. In certain embodiments, the method further comprises a method of treating a non-cancerous disease or hyperplastic condition. The cell may be a pre-cancerous or a cancerous cell. In certain embodiments, the compositions and methods inhibit the growth of the cell, induce apoptosis in the cell, and/or inhibit the translation of an oncogene or a gene that may contribute to resistance to therapy. The siNA may inhibit the translation of a gene that is overexpressed in the cancerous cell. The gene may be PFDN1-6, in particular a PFDN4 gene or transcript thereof.

In certain embodiments, the methods of the invention further comprise administering an additional therapy to the subject. The additional therapy may comprise administering a chemotherapeutic (e.g., paclitaxel or docetaxel), a surgery, a radiation therapy, and/or a gene therapy. In certain aspects the chemotherapy is docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin, methotrexate, or combinations thereof. In certain embodiments the chemotherapy is a taxane such as docetaxal or paclitaxel. Aspects of the invention includes the administration of a cytoskeletal destabilizing agent. The chemotherapy can be delivered before, during, after, or combinations thereof relative to an inhibitor of PFDN gene transcription, processing, and/or translation, or PFDN activity. A chemotherapy can be delivered within 0, 1, 5, 10, 12, 20, 24, 30, 48, or 72 hours or more of the neutral lipid composition. A lipid composition, the second anti-cancer therapy, or both the lipid composition and the anti-cancer therapy can be administered intratumorally, intravenously, intraperitoneally, orally or by various combinations thereof.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. PFDN4 is more highly expressed in chemoresistant cell lines. Panel A: RT-PCR (22 cycles) Panel B: IP (Immunoprecipitation)/western blot analysis (α-PFDN4 specificity is demonstrated in the OVCA420 IP, in which PFDN4 binding is blocked completely by pre-incubation with immunizing peptide).

FIG. 2. Antibody specificity of α-PFDN4 in IHC. Top panel: α-PFDN4 stained ovarian tumor sample. Bottom panel: Mirror image slide of top panel stained with α-PFDN4+1 μg immunizing peptide.

FIG. 3. Representative IHC staining of ovarian tumors. Panel A: 0 faint nuclear staining Panel B: 1+ nuclear staining with no cytoplasmic staining Panel C: 2+ moderate nuclear and cytoplasmic staining. Panel D: 3+ strong nuclear and cytoplasmic staining.

FIG. 4. PFDN4 overexpression is associated with a poor outcome for ovarian cancer patients.

FIG. 5. RT-PCR siRNA mediated gene silencing of PFDN4 in SKOV3 cells.

FIG. 6. siRNA mediated silencing of PFDN4 protein expression in SKOV3 cells.

FIG. 7. Effect of PFDN4 siRNA on the microtubule cytoskeleton in SKOV3 cells 48 hours following transfection. Panels are stained for α-tubulin (Alexa 555). Panel A: Control siRNA treatment. Panel B: PFDN4 siRNA treatment.

FIG. 8. Liposomal-PFDN4 siRNA-mediated downregulation of PFDN4 is therapeutically effective for the treatment of mouse xenograft (HeyA8) tumors, both as a single agent and in combination with docetaxel.

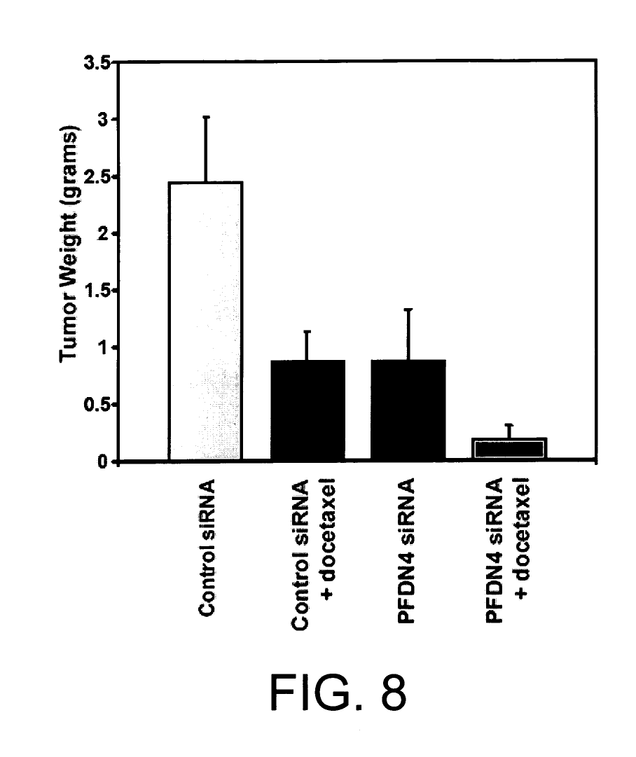

### DETAILED DESCRIPTION OF THE INVENTION

The present invention is based on the finding that PFDN4 is involved in cancer development and therapeutic resistance. For example, the inventors have found both PFDN4 mRNA and protein levels were substantially higher in ovarian cancer cells as compared to normal ovarian surface epithelium cell and there was greater PFDN4 expression in taxane-resistant cell lines (HeyA8-MDR, SKOV3-TR) as compared to their chemosensitive counterparts (HeyA8, SKOV3). Furthermore, the present invention is partly based on the finding that PFDN4 overexpression was associated with poor survival and decreased PFDN-4 expression, such as by siRNA, destabilized the cytoskeleton of ovarian cancer cells. The invention is also based on the finding that PFDN4 inhibition compliments the effects taxanes in vivo. In the present invention, PFDN4 inhibition is contemplated to be a therapeutically useful alternative for the treatment of taxane-resistant tumors.

### I. PREFOLDIN (PFDN) GENES AND PROTEINS

PFDN is a heterohexameric protein complex (PFDN1-6), which co-operates with cytosolic chaperonin CCT/TCP-1 to fold newly synthesized tubulin and actin (Hartl and hayer-Hartl, 2002; Martin et al., 2004; Geissler et al., 1998; Vainberg et al., 1998; Rommelaere et al., 2001; Martin-Benito et al., 2002; Simons et al., 2004). The PFDN4 gene maps to chromosome 20q13.2, a common site of amplification in ovarian and breast cancer (15-21). Amplification of the PFDN4 gene was first described in breast tumors and its over-expression in tumor versus normal tissue as determined by RT-PCR (Collins et al., 2001). In the case of ovarian cancer, amplifications of chromosome 20q13 are associated with poor prognosis (Suzuki et al., 2000; Gray et al., 2003; Kiechle et al., 2001; Tanner et al., 2000; Sonoda et al., 1997; Watanabe et al., 2002). The importance of PFDN subunits in normal cellular function is emphasized by their conservation throughout eukaryotic evolution. It has been demonstrated that mammalian prefoldin genes can substitute for yeast prefoldins, and rescue defects in yeast deletion mutants (Geissler et al., 1998). While deletion of prefoldins 1 through 6 are not lethal in yeast, deletion mutants grow more slowly and are supersensitive to the microtubule-depolymerizing drug, benomyl as well as the actin depolymerizing agent, latrunculin A. In C. elegans, the RNAi mediated knockdown of prefoldin subunits is embryonic lethal (Lundin and Leroux, 2005). Partial knockdown of prefoldin causes severe cytoskeletal defects including problems with meiosis, spindle assembly and cytokinesis, presumably by disrupting microtubule construction. In the present invention, the prefoldin complex as a therapeutic target for cytoskeletal disruption in the treatment of a variety of tumors, including ovarian cancer is contemplated.

### II. THERAPEUTIC GENE SILENCING

Inhibitory nucleic acid or “siNA”, as used herein, is defined as a short interfering nucleic acid. Examples of siNA include but are not limited to RNAi, double-stranded RNA, and siRNA. A siNA can inhibit the transcription or translation of a gene in a cell. A siNA may be from 16 to 1000 or more nucleotides long, and in certain embodiments from 18 to 100 nucleotides long. In certain embodiments, the siNA may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. The siNA may comprise a nucleic acid and/or a nucleic acid analog. Typically, a siNA will inhibit the processing and/or translation of a single gene within a cell; however, in certain embodiments, a siNA will inhibit the processing and/or translation of more than one gene within a cell.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90 to 100, 150, 200, 250, 300, 350, 400, 450, 500 or more contiguous nucleobases, including all ranges therebetween. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siNA (e.g., siRNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Agents of the present invention useful for practicing the methods of the present invention include, but are not limited to siRNAs of PFDN1, 2, 3, 4, 5 and/or 6. Typically, such agents are capable of (i) binding to the respective mRNA, (ii) interfere with signaling or processing and/or (iii) enhance, modulate, or augment anticancer therapy. In one embodiment, the siRNA is directed to PFDN4. The present invention provides compositions and methods using RNA interference to modulate protein expression. These methods and compositions are useful for the treatment of or contribution to the treatment of disease (e.g., cancer), induction of apoptosis, and/or interfering with biological pathways, for example cytoskeleton formation.

Typically, introduction of small interfering RNA (siRNA), induces potent and specific gene silencing, a phenomena called RNA interference or RNAi. This phenomenon has been extensively documented in the nematode C. elegans (Fire et al., 1998), but is widespread in other organisms, ranging from trypanosomes to mouse. Depending on the organism being discussed, RNA interference has been referred to as “cosuppression,” “post-transcriptional gene silencing,” “sense suppression,” and “quelling.” RNAi is an attractive biotechnological tool because it provides a means for knocking out the activity of specific genes.

Since the discovery of RNAi by Fire and colleagues in 1998, the biochemical mechanisms have been rapidly characterized. Long double stranded RNA (dsRNA) is cleaved by Dicer, which is an RNAaseIII family ribonuclease. This process yields siRNAs of ˜21 nucleotides in length. These siRNAs are incorporated into a multiprotein RNA-induced silencing complex (RISC) that is guided to target mRNA. RISC cleaves the target mRNA in the middle of the complementary region. In mammalian cells, the related microRNAs (miRNAs) are found that are short RNA fragments (−22 nucleotides). mRNAs are generated after Dicer-mediated cleavage of longer (−70 nucleotide) precursors with imperfect hairpin RNA structures. The miRNA is incorporated into a miRNA-protein complex (miRNP), which leads to translational repression of target mRNA.

In certain embodiments of the present invention, the agent for use in the methods of the present invention is a siRNA of PFDN1-6 and combinations thereof. siRNA can be used to reduce the expression level of a PFDN1-6, for example PFDN4. A siRNA of a PFDN hybridizes to a PFDN transcript or mRNA and thereby decreases or inhibits production of a PFDN protein.

In designing RNAi there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80, 85, 90, 95, 98,% or even 100% identity between the sequence of the siRNA and the gene to be inhibited. Sequences less than about 80% identical to the target gene are substantially less effective. Thus, the greater homology between the siRNA of a PFDN and a PFDN gene whose expression is to be inhibited, the less likely expression of unrelated genes will be affected.

In addition, the size of the siRNA is an important consideration. Generally, the present invention relates to siRNA molecules that are double or single stranded and comprise at least about 19-25 nucleotides, and are able to modulate the gene expression of a PFDN nucleic acid. In the context of the present invention, the siRNA is preferably less than 500, 200, 100, 50 or 25 nucleotides in length. More preferably, the siRNA is from about 19 nucleotides to about 25 nucleotides in length.

To improve the effectiveness of siRNA-mediated gene silencing, guidelines for selection of target sites on mRNA have been developed for optimal design of siRNA (Soutschek et al., 2004; Wadhwa et al., 2004). These strategies may allow for rational approaches for selecting siRNA sequences to achieve maximal gene knockdown.

While traditional antisense oligonucleotides and siRNAs are very selective with regard to gene-targeting, growing data suggest that either off-target (Jackson et al., 2003) or immune-activating effects (Kim et al., 2004; Samuel, 2004) can occur. The interferon system is highly sensitive to the presence of double-stranded RNA (dsRNA). Recent studies suggest that siRNAs synthesized using phage RNA polymerases, but not chemically synthesized siRNAs can trigger a potent induction of interferon in a variety of cell lines (Schifflelers et al., 2004; Jackson et al., 2003; Kim et al., 2004).

Several research groups have developed modifications such as chemically stabilized siRNAs with partial phosphorothioate backbone and 2′-0-methyl sugar modifications or boranophosphate siRNAs (Leung and Whittaker, 2005). Elmen and colleagues modified siRNAs with the synthetic RNA-like high affinity nucleotide analogue, Locked Nucleic Acid (LNA), which significantly enhanced the serum half-life of siRNA and stabilized the structure without affecting the gene-silencing capability (Elmen et al., 2005). Alternative approaches including chemical modification (conjugation of cholesterol to the 3′ end of the sense strand of siRNA by means of a pyrrolidine linker) may also allow systemic delivery without affecting function (Soutschek et al., 2004). Aspects of the present invention can use each of these modification strategies in combination with the compositions and methods described.

In one aspect, the invention generally features an isolated siRNA molecule of at least 19 nucleotides, having at least one strand that is substantially complementary to at least ten but no more than thirty consecutive nucleotides of a PFDN nucleic acid, and that reduces the expression of a PFDN gene or protein. In a preferred embodiment of the present invention, the siRNA molecule has at least one strand that is substantially complementary to at least ten but no more than thirty consecutive nucleotides of the mRNA for human PFDN. Each Genbank accession provided herein is incorporated herein by reference in its entirety, as of the filing date of this application. In still a further aspect the isolated siRNA molecule has at least one strand that is substantially complementary to at least 19 to 25 contiguous nucleotides of a PFDN gene or nucleic acid.

In another preferred embodiment, the siRNA molecule of a PFDN gene or nucleic acid includes a sequence that is at least 75, 80, 81, 82, 83, 84, 85, 86, 87, 88, 89, 90, 91, 92, 93, 94, 95, 96, 97, 98, 99 or 100% identity, preferably 95%, 99%, or 100% identity, to at least 10, 20, 50, 100, or 200 contiguous nucleotides of the nucleic acid sequences of a PFDN. Without undue experimentation and using the disclosure of this invention, it is understood that additional siRNAs that modulate PFDN expression can be designed and used to practice the methods of the invention.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 20040019001 and U.S. Pat. No. 6,673,611 (each of which is incorporated by reference in its entirety). Collectively, all such altered nucleic acids or RNAs described above are referred to as modified siRNAs.

Preferably, RNAi is capable of decreasing the expression of a PFDN gene or protein in a cell by at least 10%, 20%, 30%, or 40%, more preferably by at least 50%, 60%, or 70%, and most preferably by at least 75%, 80%, 90%, 95% or more.

Introduction of siRNA into cells can be achieved by methods known in the art, including for example, microinjection, electroporation, or transfection of a vector comprising a nucleic acid from which the siRNA can be transcribed. Alternatively, a siRNA can be directly introduced into a cell in a form that is capable of binding to target mRNA transcripts. To increase durability and membrane-permeability the siRNA may be combined or modified with liposomes, poly-L-lysine, lipids, cholesterol, lipofectine or derivatives thereof. In certain aspects cholesterol-conjugated siRNA can be used (see, Song et al., 2003).

### III. LIPID PREPARATIONS

To facilitate the entry of siRNA into cells and tissues, a variety of vectors including plasmids and viral vectors such as adenovirus, lentivirus, and retrovirus have been used (Wadhwa et al., 2004). While many of these approaches are successful for in vitro studies, in vivo delivery poses additional challenges based on the complexity of the tumor microenvironment. The present invention provides methods and compositions for associating an inhibitory nucleic acid, such as a siNA (e.g., a siRNA) targeting a nucleic acid sequence encoding a PFDN with a lipid and/or liposome.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. An example is the lipid dioleoylphosphatidylcholine (DOPC).

In certain embodiments of the invention, the lipid may be associated with a hemaglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer of a polynucleotide in vitro and in vivo, then they are applicable for the present invention.

A. Phospholipids

Lipid compositions of the present invention may comprise phospholipids. In certain embodiments, a single kind or type of phospholipid may be used in the creation of lipid compositions such as liposomes (e.g., DOPC used to generate neutral liposomes). In other embodiments, more than one kind or type of phospholipid may be used.

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes or lipid compositions. In certain embodiments, a lipid that is not a phospholipid (e.g., a cholesterol) can also be used.

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used in certain embodiments as the primary phosphatide (i.e., constituting 50% or more of the total phosphatide composition) because this may result in instability and leakiness of the resulting liposomes.

B. Liposomes

Liposomes are a form of nanoparticles that are attractive carriers for delivering a variety of drugs into the diseased tissue. “Liposome” is a generic term encompassing a variety of unilamellar, multilamellar, and multivesicular lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as non-uniform aggregates of lipid molecules. Also contemplated are lipofectamine-nucleic acid complexes. Liposome-mediated polynucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

Optimal liposome size depends on the tumor target. In tumor tissue, the vasculature is discontinuous, and pore sizes vary from 100 to 780 nm (Siwak et al., 2002). By comparison, pore size in normal vascular endothelium is <2 nm in most tissues, and 6 nm in post-capillary venules. Most liposomes are 65-125 nm in diameter.

Negatively charged liposomes were believed to be more rapidly removed from circulation than neutral or positively charged liposomes; however, recent studies have indicated that the type of negatively charged lipid affects the rate of liposome uptake by the reticulo-endothelial system (RES). For example, liposomes containing negatively charged lipids that are not sterically shielded (phosphatidylserine, phosphatidic acid, and phosphatidylglycerol) are cleared more rapidly than neutral liposomes.

Interestingly, cationic liposomes (1,2-dioleoyl-3-trimethylammonium-propane [DOTAP]) and cationic-liposome-DNA complexes are more avidly bound and internalized by endothelial cells of angiogenic blood vessels via endocytosis than anionic, neutral, or sterically stabilized neutral liposomes (Thurston et al., 1998; Krasnici et al., 2003). Cationic liposomes may not be ideal delivery vehicles for tumor cells because surface interactions with the tumor cells create an electrostatically derived binding-site barrier effect, inhibiting further association of the delivery systems with tumor spheroids (Kostarelos et al., 2004).

However, neutral liposomes appear to have better intratumoral penetration. Lipids with neutral or lipid compositions with a neutralized charge, e.g., 1,2-dioleoyl-sn-glycero-3-phosphatidylcholine (DOPC), can be used in various non-limiting aspects of the invention because of the neutral properties and success in delivering antisense oligonucleotides in vivo. Highly-efficient and efficacious in vivo siRNA delivery using neutral liposomes has been demonstrated in an orthotopic model of advanced ovarian cancer (Landen et al., 2005, which is incorporated herein by reference in its entirety).

The siNA may be encapsulated in the aqueous interior of a liposome, interspersed within the lipid bilayer of a liposome, attached to a liposome via a linking molecule that is associated with both the liposome and the polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The liposome or liposome/siNA associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

“Neutral liposomes or lipid composition” or “non-charged liposomes or lipid composition,” as used herein, are defined as liposomes or lipid compositions having one or more lipids that yield an essentially-neutral, net charge (substantially non-charged). By “essentially neutral” or “essentially non-charged”, it is meant that few, if any, lipids within a given population (e.g., a population of liposomes) include a charge that is not canceled by an opposite charge of another component (e.g., fewer than 10% of components include a non-canceled charge, more preferably fewer than 5%, and most preferably fewer than 1%). In certain embodiments of the present invention, a composition may be prepared wherein the lipid component of the composition is essentially neutral but is not in the form of liposomes.

In certain embodiments, neutral liposomes or lipid compositions may include mostly lipids and/or phospholipids that are themselves neutral. In certain embodiments, amphipathic lipids may be incorporated into or used to generate neutral liposomes or lipid compositions. For example, a neutral liposome may be generated by combining positively and negatively charged lipids so that those charges substantially cancel one another. For such a liposome, few, if any, charged lipids are present whose charge is not canceled by an oppositely-charged lipid (e.g., fewer than 10% of charged lipids have a charge that is not canceled, more preferably fewer than 5%, and most preferably fewer than 1%). It is also recognized that the above approach may be used to generate a neutral lipid composition wherein the lipid component of the composition is not in the form of liposomes.

In certain embodiments, a neutral, positive, or negative liposome may be used to deliver a siRNA. The liposome may contain a siRNA directed to the suppression of translation of a single gene, or the neutral liposome may contain multiple siRNA that are directed to the suppression of translation of multiple genes, e.g., one or more prefoldin (PFDN) genes or transcripts. Further, the liposome may also contain a chemotherapeutic in addition to the siRNA; thus, in certain embodiments, chemotherapeutic and a siRNA may be delivered to a cell (e.g., a cancerous cell in a human subject) in the same or separate compositions. An advantage to using neutral liposomes is that, in contrast to the toxicity that has been observed in response to cationic liposomes, little to no toxicity has yet been observed as a result of neutral liposomes. The inventors contemplate using neutral, positive, or negative lipids or liposomes to deliver inhibitors of PFDN nucleic acid processing.

C. Production of Liposomes

Liposomes and lipid compositions of the present invention can be made by different methods. For example, a nucleotide (e.g., siRNA) may be encapsulated in a liposome using a method involving ethanol and calcium (Bailey and Sullivan, 2000). The size of the liposomes varies depending on the method of synthesis. A liposome suspended in an aqueous solution is generally in the shape of a spherical vesicle, and may have one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Lipids suitable for use according to the present invention can be obtained from commercial sources. For example, dimyristyl phosphatidylcholine (“DMPC”) can be obtained from Sigma Chemical Co., dicetyl phosphate (“DCP”) can be obtained from K & K Laboratories (Plainview, N.Y.); cholesterol (“Chol”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the only solvent since it is more readily evaporated than methanol.

Liposomes within the scope of the present invention can be prepared in accordance with known laboratory techniques. In certain embodiments, liposomes are prepared by mixing liposomal lipids, in a solvent in a container (e.g., a glass, pear-shaped flask). The container will typically have a volume ten-times greater than the volume of the expected suspension of liposomes. Using a rotary evaporator, the solvent may be removed at approximately 40° C. under negative pressure. The solvent may be removed within about 5 minutes to 2 hours, depending on the desired volume of the liposomes. The composition can be dried further in a desicator under vacuum. Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

Liposomes can also be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE (1979), the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

Dried lipids or lyophilized liposomes may be dehydrated and reconstituted in a solution of inhibitory peptide and diluted to an appropriate concentration with a suitable solvent (e.g., DPBS). The mixture may then be vigorously shaken in a vortex mixer. Unencapsulated nucleic acid may be removed by centrifugation at 29,000 g and the liposomal pellets washed. The washed liposomes may be resuspended at an appropriate total phospholipid concentration (e.g., about 50-200 mM). The amount of nucleic acid encapsulated can be determined in accordance with standard methods. After determination of the amount of nucleic acid encapsulated in the liposome preparation, the liposomes may be diluted to appropriate concentrations and stored at 4° C. until use.

### IV. NUCLEIC ACIDS

The present invention provides methods and compositions for the delivery of siNA. Because a siNA is composed of a nucleic acid, methods relating to nucleic acids (e.g., production of a nucleic acid, modification of a nucleic acid, etc.) may also be used with regard to a siNA.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may formed by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “ss” and a double stranded nucleic acid by the prefix “ds”.

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Purine and pyrmidine derivatives or analogs include, but are not limited to (abbreviation/modified base description): ac4c/4-acetylcytidine, Mam5s2u/5-methoxyaminomethyl-2-thiouridine, Chm5u/5-(carboxyhydroxylmethyl) uridine, Man q/Beta, D-mannosylqueosine, Cm/2′-O-methylcytidine, Mcm5s2u/5-methoxycarbonylmethyl-2-thiouridine, Cmnm5s2u/5-carboxymethylamino-methyl-2-thioridine, Mcm5u/5-methoxycarbonylmethyluridine, Cmnm5u/5-carboxymethylaminomethyluridine, Mo5u/5-methoxyuridine, D/Dihydrouridine, Ms2i6a, 2-methylthio-N-6-isopentenyladenosine, Fm/2′-O-methylpseudouridine, Ms2t6a/N-((9-beta-D-ribofuranosyl-2-methylthiopurine-6-yl)carbamoyl)threonine, Gal q/Beta,D-galactosylqueo sine, Mt6a/N-((9-beta-D-ribofuranosylpurine-6-yl)N-methyl-carbamoyl)threonine, Gm/2′-O-methylguanosine, Mv/Uridine-5-oxyacetic acid methylester, I/Inosine, o5u/Uridine-5-oxyacetic acid (v), 16a/N6-isopentenyladenosine, Osyw/Wybutoxosine, m1a/1-methyladenosine, P/Pseudouridine, m1f/1-methylpseudouridine, Q/Queosine, m1g/1-methylguanosine, s2c/2-thiocytidine, m1I/1-methylinosine, s2t/5-methyl-2-thiouridine, m22g/2,2-dimethylguanosine, s2u/2-thiouridine, m2a/2-methyladenosine, s4u/4-thiouridine, m2g/2-methylguanosine, T/5-methyluridine, m3c/3-methylcytidine, t6a/N-((9-beta-D-ribofuranosylpurine-6-yl)carbamoyl)threonine, m5c/5-methylcytidine, Tm/2′-β-methyl-5-methyluridine, m6a/N6-methyladenosine, Um/2′-O-methyluridine, m7g/7-methylguanosine, Yw/Wybutosine, Mam5u/5-methylaminomethyluridine, or X/3-(3-amino-3-carboxypropyl)uridine, (acp3)u.

A nucleobase may be comprised in a nucleside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides, or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as flourescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR™, to reduce false positives and discriminate between single base mutants, as described in U.S. Pat. No. 5,891,625. Other modifications and uses of nucleic acid analogs are known in the art, and it is anticipated that these techniques and types of nucleic acid analogs may be used with the present invention. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

F. Preparation of Nucleic Acids

A nucleic acid may be made by any technique known to one of ordinary skill in the art, such as chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

G. Purification of Nucleic Acids

A nucleic acid may be purified on polyacrylamide gels, cesium chloride centrifugation gradients, or by any other means known to one of ordinary skill in the art (see for example, Sambrook et al., 2001, incorporated herein by reference).

In certain embodiments, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

H. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

### V. CANCER

The present invention may be used to treat a disease, such as cancer. For example, a siRNA may be delivered to treat a cancer. The cancer may be a solid tumor, metastatic cancer, or non-metastatic cancer. In certain embodiments, the cancer may originate in the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In certain embodiments, the cancer is human ovarian cancer. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease).

### VI. PHARMACEUTICAL PREPARATIONS

Where clinical application of a composition comprising a siNA is undertaken, it will generally be beneficial to prepare the composition as a pharmaceutical composition appropriate for the intended application. This will typically entail preparing a pharmaceutical composition that is essentially free of pyrogens, as well as any other impurities that could be harmful to humans or animals. One may also employ appropriate buffers to render the complex stable and allow for uptake by target cells.

The phrases “pharmaceutical or pharmacologically acceptable” refers to molecular entities and compositions that do not produce an adverse, allergic or other untoward reaction when administered to an animal, such as a human, as appropriate. The preparation of a pharmaceutical composition that contains at least one non-charged lipid component comprising a siNA or additional active ingredient will be known to those of skill in the art in light of the present disclosure, as exemplified by Remington: The Science and Practice of Pharmacy, 21st, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 μg/kg/body weight to about 100 mg/kg/body weight, about 5 microgram/kg/body weight to about 500 milligram/kg/body weight, etc., can be administered.

A gene expression inhibitor may be administered in a dose of 1, 2, 3, 4, 5, 6, 7, 8, 9, 10, 15, 20, 25, 30, 40, 50, 60, 70, 80, 90, 100 or more μg of nucleic acid per dose. Each dose may be in a volume of 1, 10, 50, 100, 200, 500, 1000 or more μl or ml.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### VII. COMBINATION TREATMENTS

In certain embodiments, the compositions and methods of the present invention involve an inhibitor of gene expression, or construct capable of expressing an inhibitor of gene expression, in combination with a second or additional therapy. The methods and compositions including combination therapies enhance, modulate, or augment a therapeutic effect, and/or increase the therapeutic effect of another anti-cancer or anti-hyperproliferative therapy, for example enhancing sensitivity of a target cell to an anticancer therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an inhibitor of gene expression and a second therapy. A tissue, tumor, or cell can be contacted with one or more compositions or pharmacological formulation(s) including one or more of the agents (i.e., inhibitor of gene expression or an anti-cancer agent), or by contacting the tissue, tumor, and/or cell with two or more distinct compositions or formulations, wherein one composition provides (1) an inhibitor of gene expression; (2) an anti-cancer agent, or (3) both an inhibitor of gene expression and an anti-cancer agent. Also, it is contemplated that such a combination therapy can be used in conjunction with a chemotherapy, radiotherapy, surgical therapy, or immunotherapy.

An inhibitor of gene expression (e.g., siNA) may be administered before, during, after or in various combinations relative to an anti-cancer treatment. The administrations may be in intervals ranging from concurrently to minutes to days to weeks. In embodiments where the inhibitor of gene expression is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the inhibitor of gene expression therapy and the anti-cancer therapy within about 12 to 24 or 72 h of each other and, more preferably, within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed. For the example below an inhibitor of gene expression therapy is “A” and an anti-cancer therapy is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of any compound or therapy of the present invention to a patient will follow general protocols for the administration of such compounds, taking into account the toxicity, if any, of the agents. Therefore, in some embodiments there is a step of monitoring toxicity that is attributable to combination therapy. It is expected that the treatment cycles would be repeated as necessary. It also is contemplated that various standard therapies, as well as surgical intervention, may be applied in combination with the described therapy.

In specific aspects, it is contemplated that a standard therapy will include chemotherapy, radiotherapy, immunotherapy, surgical therapy or gene therapy and may be employed in combination with the inhibitor of gene expression therapy, anticancer therapy, or both the inhibitor of gene expression therapy and the anti-cancer therapy, as described herein.

A. Chemotherapy

Cancer therapies include a variety of combination therapies with both chemical and radiation based treatments. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate, or any analog, derivative, or variant of the foregoing. In one aspect the chemotherapy is a cytoskeleton and/or microtubule modulating agent.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves, proton beam irradiation (U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

C. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

D. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VIII. KITS AND DIAGNOSTICS

In various aspects of the invention, a kit is envisioned containing therapeutic agents and/or other therapeutic and delivery agents. In some embodiments, the present invention contemplates a kit for preparing and/or administering a therapy of the invention. The kit may comprise reagents capable of use in administering an active or effective agent(s) of the invention. Reagents of the kit may include at least one inhibitor of gene expression, one or more lipid component, one or more anti-cancer component of a combination therapy, as well as reagents to prepare, formulate, and/or administer the components of the invention or perform one or more steps of the inventive methods.

In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods, and will follow substantially the same procedures as described herein or are known to those of ordinary skill.

### IX. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

Tissues and Cell Lines: All of the samples were collected in compliance with requirements of the M.D. Anderson Cancer Center Institutional Review Board for the Protection of Human Subjects. Tumor tissue was harvested immediately following surgical procedures, snap frozen under liquid nitrogen and then stored at −80° C. prior to RNA extraction. For immunohistochemical studies, tumor samples were formalin-fixed and paraffin embedded using standard histological techniques. All tumors were surgically staged according to International Federation of Gynecology and Obstetrics criteria. Formalin-fixed paraffin-embedded sections of ovarian tumors were obtained from the M.D. Anderson Cancer Center Department of Pathology files. HeyA8, Hey A8-MDR, SK-OV3ip1 and SK-OV3ip1-TR were obtained from Dr. Anil Sood (MDACC, Houston, Tex.). OVCAR5, OVCAR8, TOV-112D, TOV-21G, OV90 ovarian cancer cell lines were obtained from Dr. Joe Gray (UCSF, San Francisco, Calif.). DOV13 cells were obtained from Dr. Gordon Mills (MDACC, Houston, Tex.). MCF-7, PA-1, OVCAR3, and ES-2 ovarian cancer cell lines were obtained from the ATCC. OVCA420, OVCA429, OVCA432, OVCA433 were obtained from Dr. Robert Bast (MDACC, Houston, Tex.). MDA2774 was obtained from Dr. Ralph Freedman (MDACC, Houston, Tex.). The A2780 line was developed in the lab of Stuart Aaronson at the National Cancer Institute, Bethesda, Md. Cell lines were maintained in RPMI 1640 supplemented with 10% fetal bovine serum and 2 mM L-glutamine at in 5% CO₂-95% air at 37° C. Immortalized normal ovarian epithelial cells (IOSE29) were obtained from Dr. N. Auersperg (University of British Columbia, Vancouver, Canada) and were cultured in NOE media (1:1 mixture of Medium-199 and MCDB-105, supplemented with 15% fetal bovine serum, 2 mM L-glutamine, 10 ng/mL EGF, 10⁵ U/mL penicillin G).

RT-PCR: RT-PCR RNA was extracted from ovarian cancer cell lines using TRIzo10 (Invitrogen, Carlsbad, Calif.) as per manufacturer's instructions. 2 μg of total mRNA was reverse transcribed using the Superscript first strand kit (Superscript, Invitrogen, Carlsbad, Calif.), and 2 μL from each reaction was subjected to PCR using standard techniques. PFDN4 primers generated a 413 by product (PFDN4 forward: CCCAAGATGGCGGCCACCATGAAG (SEQ ID NO:1); PFDN4 reverse: GTTTAACTTTCATCAGCTTCAAGG (SEQ ID NO:2)). GAPDH primers generated an approximately 800 by product (GAPDH forward:TGAAGGTCGGAGTCAACGGATTTGGT (SEQ ID NO:3); GAPDH reverse: CATGTGGGCCATGAGGTCCACCAC (SEQ ID NO:4)). Semi-quantitative cycling conditions were optimized to the following program: 98° C. for 2 minutes followed by 22 cycles at 95° C. for 30 seconds, 55° C. for 30 seconds and 72° C. for 60 seconds, followed by a final cycle at 72° C. for 5 minutes. PCR products were visualized by agarose gel electrophoresis.

Antibodies: Antibodies anti-PFDN4 polyclonal antibodies were generated in rabbits against the C-terminal 14 amino acids of PFDN4 (AKFGSNINLEADES (SEQ ID NO:5)) by Sigma-Genosys (Woodlands, Tex.). A Sulfolink (Pierce, Rockford, Ill.) PFDN4-peptide column was used to affinity-purify antibodies for immunohistochemical studies. PFDN4 antibodies were used at a concentration of 2.5 μg/mL for immunohistochemical studies and at 0.5 μg/mL for western blot analysis. Anti-GAPDH antibodies (Ambion, Austin, Tex.) were used to monitor equal protein loading in western blot analysis of whole cell lysates. Anti-α-tubulin monoclonal antibodies utilized for indirect immunofluorescent staining of the microtubule cytoskeleton were purchased from Sigma (St. Louis, Mo.) and were used at a 1:2000 dilution. Alexafluor 555-labeled goat-anti-mouse secondary antibodies used in these studies were purchased from Invitrogen (Carlsbad, Calif.),

Immunohistochemistry: Immunohistochemical staining was performed on 5 μm paraffin-embedded sections using the Universal DAKO Labeled Streptavidin-Biotin 2 System (DAKO LSAB2 System, horseradish peroxidase (HRP), Dako Corp., Carpinteria, Calif.), as per the manufacturer's instructions. Briefly, sections were deparaffinized in xylene and rehydrated in a decreasing gradient of ethanol in water. Antigen retrieval was subsequently performed in a pressure cooker with 0.01 M citrate buffer, pH 6.0, for 20 minutes. Hydrogen peroxide (0.3%) was applied to quench the endogenous peroxidase activity. The slides were then incubated in protein blocking agent to reduce nonspecific binding. The sections were incubated with primary antibodies for 1 hour. The sections were then washed in phosphate-buffered saline (PBS) to remove unbound primary antibody, and incubated with a biotinylated secondary antibody. The sections were washed in PBS and staining was completed by incubation with streptavidin-HRP and 3,3′-diaminobenzidine colorimetric reagents. Finally, sections were counter-stained with hematoxylin. The intensity of the immunostaining was graded as negative (no staining), weak (1+), moderate (2+) or strong (3+). Tumors with 2+ or 3+ staining in greater than 10% of the tumor cells were considered positive. Antibody specificity was demonstrated by preincubating PFDN4 antibodies with 1 μg of immunizing peptide, prior to application to the tissue section.

Immunoprecipitation and Western Blot Analysis: Each 10 cm plate of cells was lysed in 1 mL NP-40 lysis buffer (50 mM Hepes, pH 7.25, 150 mM NaCl, 100 mM ZnCl₂, 50 mM NaF, 2 mM EDTA, 1 mM sodium orthovanadate, 1% NP-40), supplemented with Roche complete protease inhibitors (Roche Diagnostics, Indianapolis, Ind.). Cell lysates were cleared by centrifugation at 14000 g for 15 minutes at 4° C. Protein concentration was determined using the BCA Protein Assay (Pierce Biotechnology, Rockford, Ill.). 1 mg total protein lysate was combined with 50 μL of 20% Protein A Sepharose CL4B beads (Amersham Biosciences, Piscataway, N.J.) and 1 μg of anti-PFDN4 antibody. The mix was incubated with gentle rocking for 1.5 hours at 4° C. and washed 3 times in NP40 lysis buffer prior to resuspension in 25 μL Laemmli sample buffer. Immunoprecipitating proteins were resolved by 15% SDS-PAGE, electrophoretically transferred to Immobilon P membrane (Millipore, Bedford, Mass.) and subjected to western blot analysis. The blot was blocked for 1 hour in 5% milk powder in TBST (10 mM Tris, pH 8, 150 mM NaCl, 0.05% Tween-20). Primary antibodies were added to a concentration of 0.5 μg/mL and the blot was further incubated at 4° C. overnight. The blot was washed five times for five minutes in TBST and prior to the addition of HRP-conjugated secondary antibodies for 1 hour. Immunoreactive proteins were visualized using enhanced chemiluminescence (ECL, Amersham Biosciences, Piscataway, N.J.). For western blots of whole cell lysates (50 μg total protein), anti-GAPDH was used to confirm equal protein loading per lane.

RNA interference: PFDN4 siRNA (sense: UUCAGCGAGUGUUAGCAGATT (SEQ ID NO:6); antisense: UCUGCUAACACCGCUGAATT (SEQ ID NO:7)) and AllStars Negative Control siRNA were purchased from Qiagen (Valencia, Calif.). HeyA8 and SKOV3 were grown to 50% confluence prior to transfection with control or PFDN4 siRNA using Lipofectamine 2000 (Invitrogen, Valencia, Calif.) following manufacturer's instruction. Cells were typically harvested for analysis 48-72 hours following transfection. Immunofluorescent staining HeyA8 and SKOV3 cells were grown on glass coverslips in 24-well plates for 24 hours prior to transfection with either control or PFDN4 siRNA, and cultured for an additional 48 hours. Media was then aspirated and replaced with 0.5 mL of 4% paraformaldehyde for 30 minutes. Cells were then incubated with 100 mM glycine for 5 minutes, washed once with PBS and then incubated with 0.2% TritonX-100 in PBS for 10 minutes. Coverslips were washed thrice in PBS and then blocked for 30 minutes in 10% goat serum in PBS. Coverslips were then incubated with a 1:2000 dilution of anti-α-tubulin antibodies for 1.5 hours at room temperature. Slides were washed three times with PBS and then incubated for 1 hour with Alexa 555-labelled anti-rabbit secondary antibodies. Coverslips were washed twice with PBS and finally mounted in anti-fade mounting media and then visualized by fluorescence microscopy.

Animal Studies: Female athymic nude mice (NCr-nu) were purchased from the National Cancer Institute-Frederick Cancer Research and Development Center (Frederick, Md.). Animal experiments were conducted with the approval of the M.D. Anderson Animal Care and Use Committee, and in accordance with American Association for Accreditation of Laboratory Animal Care and the USPHS “Policy on Human Care and Use of Laboratory Animals”. Xenograft tumors were typically established by intraperitoneal injection of 250,000 Hey A8 cells. The inventors have previously demonstrated that this model exhibits the intra-abdominal growth pattern of advanced ovarian cancer (Landen et al., 2005; Landen et al., 2006; Halder et al., 2006). For therapy experiments, tumors were initiated in 40 mice and were allowed to establish for one week prior to the start of therapy. Mice were then divided into four treatment groups (10 per group) and treated with the following agents: control liposomal siRNA; control siRNA and docetaxel; PFDN4 liposomal siRNA; PFDN4 siRNA and docetaxel. Liposomal siRNA (5 μg per mouse) was prepared as previously described (Landen et al., 2005; Landen et al., 2006; Halder et al., 2006) and administered intraperitoneally twice weekly in 200 μL of normal saline. Docetaxel (50 μg/mouse) was injected intraperitoneally once weekly in 100 μL of normal saline. Mice were sacrificed following 2.5 weeks of therapy. Mouse weight, tumor weight, and distribution of tumor were recorded. Tumor tissue samples were snap frozen as well as formalin-fixed and paraffin-embedded for molecular analysis. Statistics: To evaluate differences in overall survival based on PFDN4 expression, the Kaplan-Meier method was used to generate survival curves and the log-rank test was used to compare differences. A p-value of less than 0.05 was considered statistically significant.

### Example 2

### Elevated PFDN4 Expression in Chemoresistant Cancer Cells

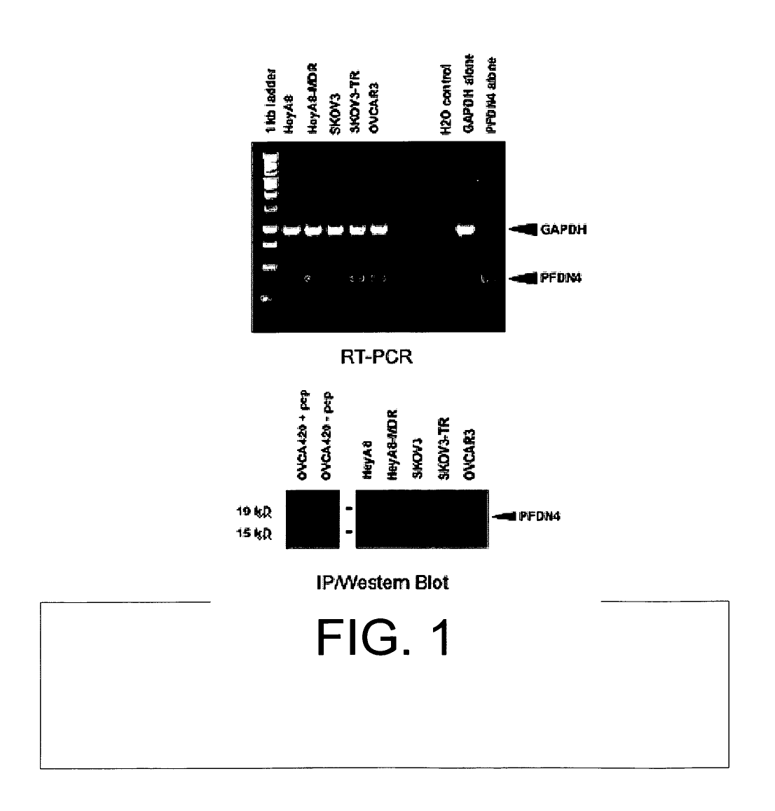

PFDN4 is more highly expressed in chemoresistant ovarian cancer cell lines. Twenty-five ovarian cancer cell lines were screened for expression levels of PFDN4 using both RT-PCR and western blot analysis. Perhaps not surprisingly, because of the active cytoskeletal construction in dividing cells, PFDN4 was universally expressed by all cell lines tested (data not shown). Of note, PFDN4 mRNA and protein levels were considerably lower in the comparatively slow-growing normal ovarian surface epithelium (HIO-180) as compared to ovarian cancer cell lines. Interestingly, as seen in FIG. 1A, increased levels of PFDN4 mRNA were detected in the chemoresistant HeyA8-MDR and SKOV3-TR cell lines (IC50 docetaxel >=250 nM for both) as compared to their chemosensitive parental counterparts HeyA8 and SKOV3 (IC50 docetaxel=1-6.2 nM). This is reflected in a modest increase in PFDN4 protein production as determined by immunoprecipitation and western blot analysis (FIG. 1B) in both HeyA8-MDR and SKOV3-TR. Interestingly, OVCAR-3 is a drug-resistant cell line, and also expresses detectably more PFDN4 mRNA as compared to the chemosensitive parental HeyA8 and SKOV3 cell lines. It is possible that PFDN may be upregulated as a compensatory mechanism, which is intended to increase tubulin synthesis as a defensive response to taxane treatment.

### Example 3

### PFDN4 Expression Levels in Ovarian Tumors Correlates with Poor Patient Outcome

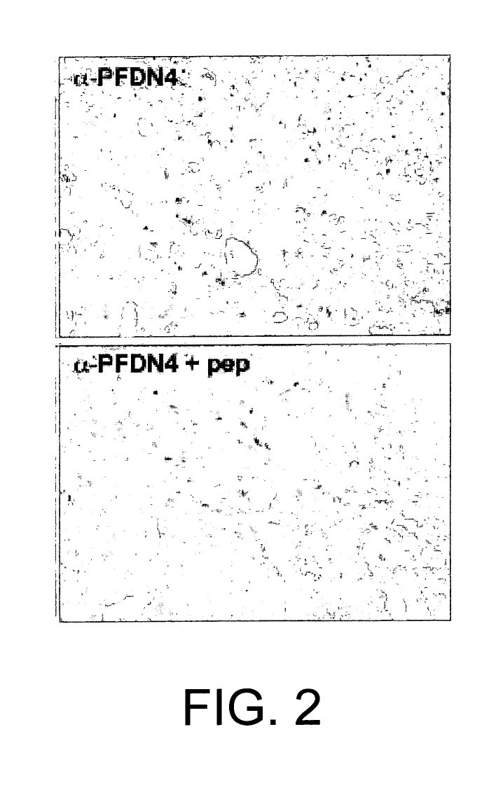

PFDN4 Expression levels in Ovarian Tumors correlates with poor patient outcome Formalin-fixed paraffin-embedded sections from 68 ovarian cancer patients were obtained through the M.D. Anderson Cancer Center Department of Pathology files and the M.D. Anderson Gynecologic Tumor Bank. For immunohistochemical staining, PFDN4 antibodies were used at a concentration of 2.5 μg/mL and immunoreactivity could be fully blocked by pre-incubation for 15 minutes with the PFDN4 immunizing peptide (FIG. 2), which confirmed the specificity of the antibody.

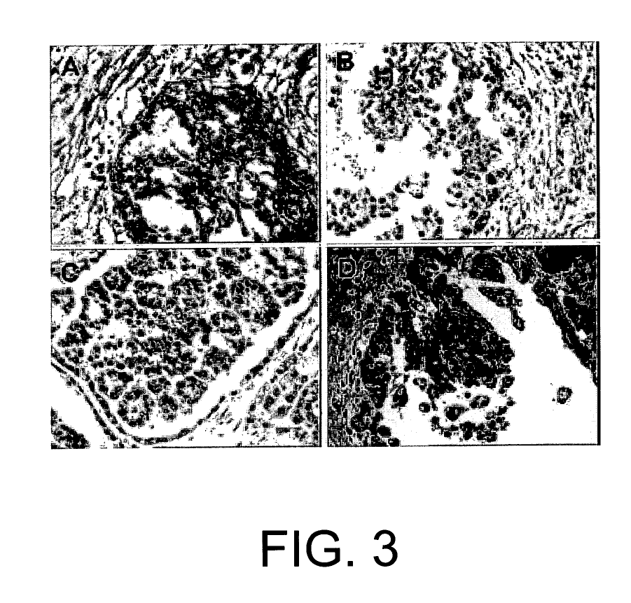

Obvious differences were observed in PFDN4 staining intensity and subcellular localization among tumor samples (FIG. 3). Typically, any single tumor stained uniformly for PFDN4 with little intra-tumor variation. Interestingly, PFDN4 localized primarily to the nucleus of both tumor and normal cells (FIG. 3 panel B, C, D). Intense cytoplasmic staining was also seen in some tumors (3D).

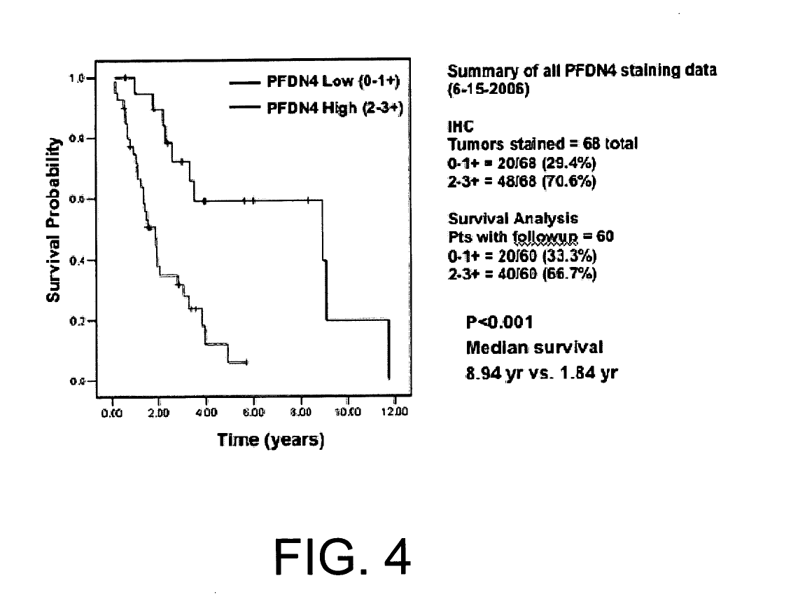

The initial analysis correlated PFDN4 staining intensity with overall patient outcome. Kaplan-Meier analysis indicated that the PFDN4 overexpression correlated with poor outcome as compared to patients with negative tumoral staining for PFDN4 (P=0.001), with median survival times of 1.84 years versus 8.94 years, respectively (FIG. 4). PFDN4 overexpression is therefore predictive of aggressive tumor behavior. Because poor outcome is frequently a consequence of acquired chemoresistance, these data support a role for the involvement of PFDN4 in taxane resistance observed in ovarian tumors.

With regards to the nuclear staining observed in both normal epithelium as well as ovarian tumors, there is evidence in the literature that PFDN4 functions independently of its role as a chaperone subunit. In 1996, PFDN4 was originally identified as “C-1” in an SV40 immortalized Wilms tumor derived fibroblast line-26. The C-1 gene was induced in the GO-S phase transition of normal cycling cells, and was believed to be a transcription factor whose expression was related to the cell cycle. Recently, PFDN4 was identified as part of another protein complex including the F-box protein SKP1; the prefoldins URI-1 (unconventional prefoldin RPB5 interactor), PFDN2, STAP-1 (SKP2 associating α-class PFDN1); RPB5 (subunit of the RNA polymerases I, II and III) and the ATPases TIP48 and TIP49. The latter three components of this complex are linked to transcription and chromosome remodeling.

Furthermore, URI-1 is implicated in the control of genomic integrity and DNA repair. The participation of PFDN4 in this complex further supports additional roles for PFDN4 in the nucleus as well as within the cytoplasm.

### Example 4

### PFDN4 SIRNA Mediated Gene Silencing Impacts Ovarian Tumor Cell Growth

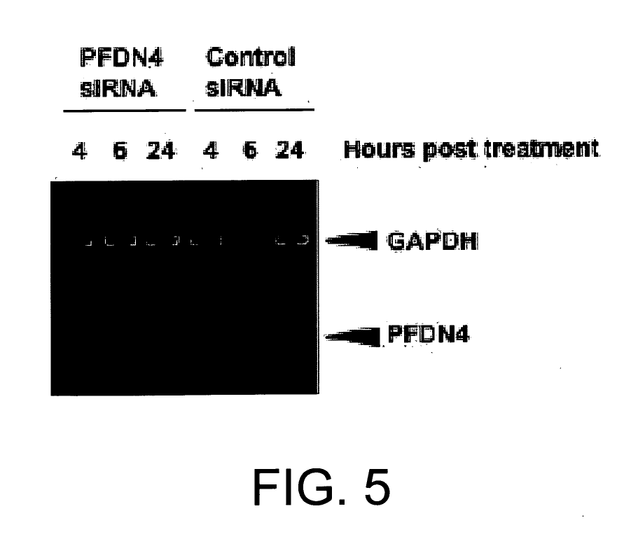

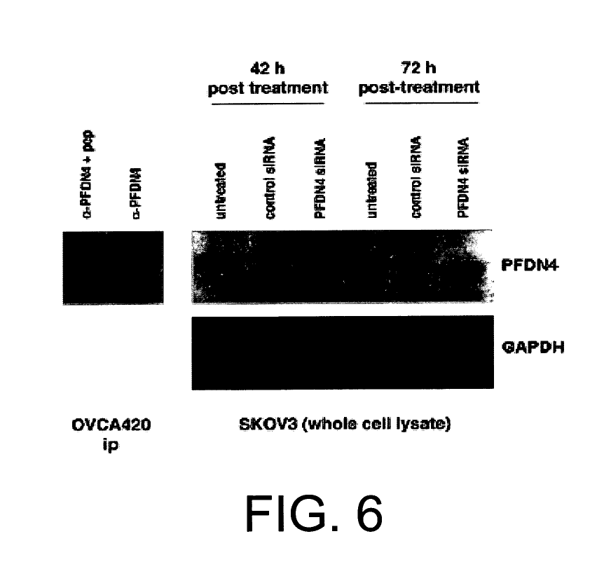

To determine the effect of PFDN4 downregulation in ovarian cancer in vitro, the inventors utilized RNA interference (RNAi) technology to silence PFDN4 expression in ovarian cancer cells. To determine the efficiency of gene silencing, they first examined PFDN4 mRNA levels at 4, 6 and 24 hours following transfection. Using an RT-PCR based screen, the inventors detected a decrease in PFDN4 mRNA within 4 hours following transfection, with greater than 90% silencing observed by 24 hours following transfection (FIG. 5). The result demonstrated a detectable down-regulation in PFDN4 protein production in vitro by 48 hours, which is suggestive PFDN4 protein stability (FIG. 6).

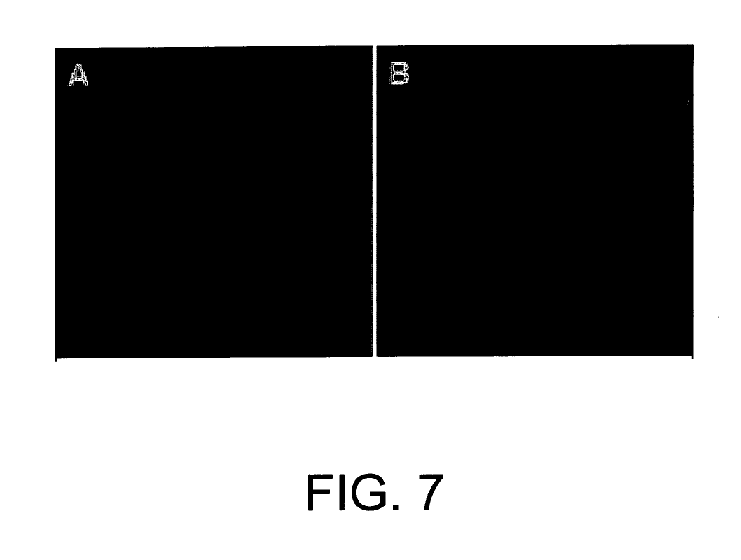

To determine the effect of PFDN4 gene silencing on the cytoskeleton of ovarian cancer cells, ovarian cancer cells grown on coverslips in 24-well plates were transfected with PFDN4 and control siRNA. The microtubule cytoskeleton of these cells was examined at 24, 48 and 72 hours following transfection, using indirect immunofluorescent staining for α-tubulin. When examined by light microscopy, both control and PFDN4 siRNA transfected cells appear normal. However, when viewed by fluorescent microscopy at 48 hours following transfection, distinct changes in the morphology microtubule cytoskeleton of PFDN4 siRNA treated cells could be observed. Specifically, microtubules of PFDN4 siRNA treated cells appeared less organized (FIG. 7B) and more condensed when compared to the well-networked cytoskeleton of control siRNA treated cells (FIG. 7A). Based on this disruption of cytoskeletal assembly, it was predicted that inhibition of PFDN4 in vivo would impact the tumor growth.

### Example 5

### Therapeutic Inhibition of PFDN4 in a Mouse Xenograft Model of Ovarian Cancer

The inventors have previously demonstrated the feasibility of therapeutic liposomal siRNA delivery to tumors in vivo and demonstrated the preferential accumulation of siRNA in intraperitoneal ovarian tumors using a neutral liposome formulation. In the present invention they used this novel technology to test the therapeutic efficacy of PFDN4 siRNA mediated silencing for the treatment of ovarian cancer.

Nude mice bearing HeyA8 intraperitoneal ovarian tumors (7 days following tumor cell injection) divided into four treatment groups (n=10 mice per group) including: control (non-silencing) siRNA-DOPC; siRNA-DOPC and docetaxel; PFDN4 siRNA-DOPC; PFDN4 siRNA-DOPC and docetaxel. Treatment was carried out for a total of 3 weeks. At the end of the test period, the mice were sacrificed and autopsied.

The average size of HeyA8 tumors in mice treated with control liposomal siRNA was 2.44 g. Strikingly, the reduction in tumor growth in mice treated with liposomal PFDN4 siRNA (PFDN4 0.874 g vs control: 2.44, p=0.009) rivaled that of docetaxel and control siRNA combination therapy (Docetaxel+control siRNA: 0.87 g vs. control: 2.44 g, p=0.002). When combined with docetaxel, PFDN4 siRNA therapy resulted in a 92.7% reduction in tumor size (0.179 g vs control: 2.44 g, p<0.001) and was statistically more effective than either PFDN4 siRNA or docetaxel alone (p=0.002). Taken together, these animal studies strongly suggest that PFDN4 represents a druggable target for the more effective treatment of ovarian tumors, both alone and in combination with conventional chemotherapeutic agents. Furthermore, because PFDN4 siRNA disrupts cytoskeletal assembly by a completely different mechanism than taxanes, it is further contemplated that PFDN4 inhibitor can be used in combination with anti-taxane therapy.

Homo sapiens prefoldin subunit 4 (PFDN4), mRNA, gi|54792079| accession number NM_{—}002623.3 Includes the nucleic acid sequence that comprises the following nucleotide

(SEQ ID NO: 8)
AAAGTCCAAGAGGACGGAATGTGGAGACAGTGTTGTATTTTTGCG
GGGAGTTCTAGGCCGACCGGGAGCGAGAGAACGCTCGGGGGCGAA
GCGCGCCATTGCGGCCCTCCCCGCCGCCTGCGGTAGTCCAGTCCC
AAGATGGCGGCCACCATGAAGAAGGCGGCTGCAGAAGATGTCAAT
GTTACTTTCGAAGATCAACAAAAGATAAACAAATTTGCACGGAAT
ACAAGTAGAATCACAGAGCTGAAGGAAGAAATAGAAGTAAAAAAG
AAACAACTCCAAAACCTAGAAGATGCTTGTGATGACATCATGCTT
GCAGATGATGATTGCTTAATGATACCTTATCAAATTGGTGATGTC
TTCATTAGCCATTCTCAAGAAGAAACGCAAGAAATGTTAGAAGAA
GCAAAGAAAAATTTGCAAGAAGAAATTGACGCCTTAGAATCCAGA
GTGGAATCAATTCAGCGAGTGTTAGCAGATTTGAAAGTTCAGTTG
TATGCAAAATTCGGGAGCAACATAAACCTTGAAGCTGATGAAAGT
TAAACATTTTATAATACTTTTTTTATTTGTTTAATAAACTTGAAT
ATTGTTTAAAATGATAATTTTCCTTCTTCAAATGACATGGAAAGC
AAAACTTTCTTTTTTAAAAATTTTCATTTATTTAATGGAAACTTG
CCCATTTTCACATGTCTGCTTATTTATTTTATATTTTTAAAAGAA
GACAGTATTCACCTATGTATTTTGCATAACGATTATATCAAGTCT
AGGGGCTTCATGTCATGTTATTAAAATCAGTTAAGCAATCTTTTA
TGTTTCTATATTATTTAGAATATTTGTTGTTGCAATTTTCACATA
AGAAAATTTAACAGTTGTGTCATGTTGTTTCTGTCTGATTTTAAT
TGCTGTCTAATGACGGGGAAAGCACGATGAAAAGATGTACAATCC
TGCATCCTTGCTTATTTCACAACTAAAGCTTTGTCATAGACTTCA
AAATATATATGTATATATTTTATTTAAATATATGTTACATATTAT
ATTTAAACATACATATTTAACATTTTTTACATATCTATCAATATC
AGAGATTTGGGTAAAAGAATGGGTAATGTTTAAACATGTGGAGGC
ATGTGGAGCTTTATACAAACAGGGCAGAACCACAGAAGAACGTTT
TAGAAACCAAGAGATGTGCAGAAAGAAATGTTTAGTGTTTTTTCG
TTTTAAATTTTAGATTTTATTTTAGTGCTTTGTAATTAATTGGGG
TTTATATTGATAAAGATGTGGAAGTTAAACAGCTATGTATGTAAA
AGTAAGGCTTATTTCTTAAATAAAGGATGCATTTCTTCCC.

The prefoldin subunit 4, gi|12408677 accession number NP_{—}002614.2, comprises the amino acids sequence MAATMKKAAAEDVNVTFEDQQKINKFARNTSRITELKEEIEVKKKQLQNLEDACDDIMLAD DDCLMIPYQIGDVFISHSQEETQEMLEEAKKNLQEEIDALESRVESIQRVLADLKVQLYAKFG SNINLEADES (SEQ ID NO:9). Also accession numbers gb|BC062671.11|gi|38571594|, dbj|AK223394.1 gi|62898348|, gb|BT019604.1| gi|54696077|, emb|BX647130.1 gi|34366158|, dbj|AK226173.1 gi|110624425|, as well as OMIM entries 604899 PREFOLDIN 5; PFDN5 Gene map locus 12q12, 603494 NNX3 PROTEIN, 604898 PREFOLDIN 4; PFDN4 Gene map locus 20q13.2, 604897 PREFOLDIN 1; PFDN1 Gene map locus 5q31, 300133 VON HIPPEL-LINDAU BINDING PROTEIN 1; VBP1 Gene map locus Xq28, 610355 PARTNER AND LOCALIZER OF BRCA2 each of which is incorporated herein by reference in their entirety.

### X. REFERENCES

The following references, to the extent that they provide exemplary procedural or other details supplementary to those set forth herein, are specifically incorporated herein by reference.
U.S. Appln. 2002/0168707
U.S. Appln. 2003/0012812
U.S. Appln. 2003/0051263
U.S. Appln. 2003/0055020
U.S. Appln. 2003/0159161
U.S. Appln. 2004/0064842
U.S. Appln. 2004/0204377
U.S. Appln. 2004/0208921
U.S. Appln. 2004/0265839
U.S. application Ser. No. 117,363
U.S. Pat. No. 4,659,774
U.S. Pat. No. 4,683,202
U.S. Pat. No. 4,816,571
U.S. Pat. No. 4,959,463
U.S. Pat. No. 5,030,453
U.S. Pat. No. 5,141,813
U.S. Pat. No. 5,214,136
U.S. Pat. No. 5,223,618
U.S. Pat. No. 5,223,618
U.S. Pat. No. 5,264,566
U.S. Pat. No. 5,378,825
U.S. Pat. No. 5,428,148
U.S. Pat. No. 5,446,137
U.S. Pat. No. 5,466,786
U.S. Pat. No. 5,470,967
U.S. Pat. No. 5,539,082
U.S. Pat. No. 5,539,082
U.S. Pat. No. 5,554,744
U.S. Pat. No. 5,574,146
U.S. Pat. No. 5,602,240
U.S. Pat. No. 5,602,244
U.S. Pat. No. 5,610,289
U.S. Pat. No. 5,614,617
U.S. Pat. No. 5,623,070
U.S. Pat. No. 5,645,897
U.S. Pat. No. 5,652,099
U.S. Pat. No. 5,670,663
U.S. Pat. No. 5,672,697
U.S. Pat. No. 5,681,947
U.S. Pat. No. 5,700,922
U.S. Pat. No. 5,705,629
U.S. Pat. No. 5,708,154
U.S. Pat. No. 5,714,331
U.S. Pat. No. 5,714,606
U.S. Pat. No. 5,719,262
U.S. Pat. No. 5,736,336
U.S. Pat. No. 5,763,167
U.S. Pat. No. 5,766,855
U.S. Pat. No. 5,773,571
U.S. Pat. No. 5,777,092
U.S. Pat. No. 5,786,461
U.S. Pat. No. 5,792,847
U.S. Pat. No. 5,858,988
U.S. Pat. No. 5,859,221
U.S. Pat. No. 5,872,232
U.S. Pat. No. 5,886,165
U.S. Pat. No. 5,891,625
U.S. Pat. No. 5,908,845
U.S. Pat. No. 5,962,016
U.S. Pat. No. 6,506,559
U.S. Pat. No. 6,573,099
U.S. Pat. No. 6,680,068
U.S. Pat. No. 4,682,195
Aguirre Ghiso, Oncogene, 21:2513-24, 2002.
Ambros et al., Nature, 431:350-355, 2004.
Apte et al., Gynecol. Oncol., 93:78-86, 2004.
Bailey and Sullivan, Biochimica. Biophys. Acts., 239-252, 2000.
Baird and Kaye, Eur. J. Cancer, 39:2450-2461, 2003.
Bangham et al., J. Mol. Biol., 13(1):253-259, 1965.
Berkenblit and Cannistra, J. Reprod. Med., 10:426-438, 2005.
Bonome et al., Cancer Res. In press 2006.
Brummelkamp et al., Cancer Cell, 2:243-247, 2002.
Cance et al., Clinical Cancer Research, 6:2417-2423, 2000.
Cannistra, N. Engl. J. Med., 351:2519-2529, 2004.
Casamassima and Rozengurt, J. Biol. Chem., 273: 26149-56, 1998.
Collins et al., Genome Res., 11:1034-1042, 2001.
Daniel et al., Kidney Int. Suppl., 57:S73-81, 1996.
Deamer and Uster, In: Liposome Preparation: Methods and Mechanisms, Ostro (Ed.), Liposomes, 1983.
Devroe and Silver, Expert Opin. Biol. Ther., 4:319-327, 2004.
Dobrzanski et al., Cancer Res., 64:910-919, 2004.
Dokka et al., Pharm Res, 17: 521-25, 2000.
Donze and Picard, Nucleic Acids Res, 30(10):e46, 2002.
Dorn et al., Nucleic Acids Res., 32:e49, 2004.
du Bois et al., J Clin Oncol, 17: 46-51, 1999.
Dubey et al., J. Drug Target, 12:257-264, 2004.
Duxbury et al., Biochem. Biophys. Res. Commun., 311:786-792, 2003.
Duxbury et al., Oncogene, 23:1448-1456, 2004.
Egholm et al., Nature, 365(6446):566-568, 1993.
Elbashir et al., Nature, 411(6836):494-498, 2001.
European Appln. 01219
European Appln. 266,032
Farhood et al., Biochim. Biophys. Act, 289-295, 1995.
Fire et al., Nature, 391(6669):806-811, 1998.
Flenniken et al., Dev. Biol., 179:382-401, 1996.
Froehler et al., Nucleic Acids Res., 14(13):5399-5407, 1986.
Gabizon, Cancer Invest., 19:424-436, 2001.
Geissler et al., EMBO J., 17:952-966, 1998.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gray et al., Gynecol. Oncol., 88(1 Pt 2): S16-21, 2003.
Gregoriadis, In: Drug Carriers in Biology and Medicine, Gregoriadis (Ed.), 287-341, 1979.
Gutierrez-Puente et al., J. Pharmacol. Exp. Ther., 291:865-869, 1999.
Halder et al., Clin. Cancer Res., 12:4916-4924, 2006.
Halder et al., Clinical Cancer Research, 11: 8829-36, 2005.
Han et al., Ann Surg Oncol, 4:264-268, 1997.
Hannon and Rossi, Nature, 431:371-378, 2004.
Hartl and Hayer-Hartl, Science, 295:1852-1858, 2002.
Hassani et al., J. Gene Med., 7(2):198-207, 2005.
Hecker et al., Cancer Research, 62:2699-2707, 2002.
Hortobagyi et al., J. Clin. Oncol., 19:3422-3433, 2001.
Hsia et al., J Cell Biol, 160:753-67, 2003.
Jackson et al., Nat. Biotechnol., 21:635-637, 2003.
Jemal et al., CA Cancer J. Clin., 55(1):10-30, 2005.
Jordan and Wilson, Nat. Rev. Cancer, 4:253-265, 2004.
Judson et al., Cancer, 86: 1551-56, 1999.
Kaneda et al., Science, 243:375-378, 1989.
Kato et al, J. Biol. Chem., 266:3361-3364, 1991.
Kiechle et al., Cancer, 91:534-540, 2001.
Kim et al., Nat. Biotechnol., 22:321-325, 2004.
Kinch et al., Clin. Exp. Metastasis, 20:59-68, 2003.
Klein et al., Gastroenterology, 125:9-18, 2003.
Kohno et al., Int J Cancer, 97:336-43, 2002.
Kornberg and Baker, DNA Replication, 2nd Ed., Freeman, San Francisco, 1992.
Kornberg et al., Invest Ophthalmol V is Sci, 45:4463-69, 2004.
Kornberg, Head Neck, 20: 634-639, 1998.
Kostarelos et al., Int J Cancer, 112: 713-21, 2004.
Landen et al., Cancer Biol. Ther., 5:1708-1713, 2006.
Landen et al., Cancer Res., 65:6910-6918, 2005.
Landen, Cancer Res, 65: 6910-18, 2005.
Langley et al., Cancer Research, 63: 2971-76, 2003.
Lewis et al., Cell, 115:787-798, 2003.
Lewis et al., Nat. Genet., 32:107-108, 2002.
Lori et al., Am. J. Pharmacogenomics, 2:245-252, 2002.
Lundin and Leroux, Intl. Worm Meeting, Wormbase WBPaper 00026281, 2005.
Martin et al., Trends Biochem. Sci., 29:455-458, 2004.
Martin-Benito et al., EMBO J., 21:6377-6386, 2002.
Matsuda et al., Proc. Natl. Acad. Sci. USA, 101:16-22, 2004.
McCaffrey et al., Nature, 418:38-39, 2002.
McGuire et al., New England Journal of Medicine, 334:1-6, 1996.
McLean et al., Expert Opin Pharmacother, 4: 227-34, 2003.
Miller et al., Biochemistry, 37(37):12875-83, 1998.
Mitra et al., Nature Reviews Molecular Cell Biology, 6: 56-68, 2005.
Mitra et al., Proc Am Assoc Cancer Res, 2005.
Nemoto et al., Pathobiology, 65:195-203, 1997.
Nicolau et al., Methods Enzymol., 149:157-176, 1987.
Noblitt et al., Cancer Gene Ther., 11:757-766, 2004.
Ogawa et al., Oncogene, 19:6043-6052, 2000.
Owens et al., Cancer Res, 55:2752-2755, 1995.
Ozols et al., Cancer Cell, 5:19-24, 2004.
Park et al., Cancer Lett., 118:153-160, 1997.
PCT Appln. WO 92/20702
PCT Appln. WO02/100435A1
PCT Appln. WO03/015757A1
PCT Appln. WO04/002453A1
PCT Appln. WO04029213A2
Pellegrini and Budman, Cancer Invest., 23:264-273, 2005.
Reich et al., Mol. Vis., 9:210-216, 2003.
Remington's Pharmaceutical Sciences, 18th Ed. Mack Printing Company, 1289-1329, 1990.
Rommelaere et al., J. Biol. Chem., 276:41023-41028, 2001.
Ryther et al., Gene Ther., 12(1):5-11, 2004.
Sambrook et al., In: Molecular cloning, Cold Spring Harbor Laboratory Press, Cold Spring Harbor, N.Y., 2001.
Schaller and Parsons, Trends in Cell Biology, 3:258-62, 1993.
Schaller et al., Mol Biol Cell, 10:3489-3505, 1999.
Schaller, Biochim Biophys Acta, 1540:1-21, 2001.
Schaller, J Endocrinol, 150:1-7, 1996.
Schaller, Trends Cell Biol, 3:258-262, 1993.
Scheit, In: Synthesis and Biological Function, Wiley-Interscience, NY, 171-172, 1980.
Schlaepfer and Hunter, Trends in Cell Biology, 8: 151-57, 1998.
Schlaepfer et al., Prog Biophys Mol Biol, 71: 435-78, 1999.
Sein et al., Oncogene, 19: 5539-42, 2000.
Sheta et al., J Natl Cancer lnst, 92: 1065-73, 2000.
Shibata et al., Cancer Res, 58: 900-903, 1998.
Sieg et al., Nat Cell Biol, 2:249-56, 2000.
Simons et al., J. Biol. Chem., 279:4196-4203, 2004.
Sioud and Sorensen, Biochem. Biophys. Res. Comm., 312:1220-1225, 2003.
Siwak et al., Clin Cancer Res, 8: 955-56, 2002.
Sledz et al., Nat. Cell Biol., 5:834-839, 2003.
Song et al., Nature Med. 9:347-351, 2003.
Sonoda et al., Genes Chromosomes Cancer, 20(4):320-328, 1997.
Sonoda et al., Journal of Biological Chemistry, 275:16309-15, 2000.
Sood et al., Am J Pathol, 165:1087-1095, 2004.
Sood et al., Cancer Biology & Therapy, 1: 511-17, 2002.
Sorensen et al., J. Mol. Biol., 327:761-66, 2003.
Soutschek et al., Nature, 432:173-178, 2004.
Spagnou et al., Biochemistry, 43:13348-13356, 2004.
Sulman et al., Genomics, 40:371-374, 1997.
Suzuki et al., Cancer Res., 60:5382-5385, 2000.
Szoka and Papahadjopoulos, Proc. Natl. Acad. Sci. USA, 75:4194-4198, 1978.
Tanner et al., Cancer Res., 6:1833-1839, 2000.
Thaker et al., 36th Annual Meeting of the Society of Gynecologic Oncologists, Miami, Fla., 2005.
Thaker et al., Clin. Cancer Res., 10:5145-5150, 2004.
Uchida et al., Mol. Ther., 10:162-171, 2004.
Vainberg et al., Cell, 93:863-873, 1998.
Voskoglou-Nomikos et al., Clin. Cancer Res., 9:4227-4239, 2003.
Walker-Daniels et al., Prostate, 41:275-80, 1999.
Watanabe et al., Jpn. J. Cancer Res., 93:1114-1122, 2002.
Wianny et al., Nat. Cell Biol., 2:70-75, 2000.
Wong et al., Gene, 10:87-94, 1980.
Xia et al., Nat. Biotechnol., 20:1006-10, 2002.
Yang et al., Oncogene, 22:5694-701, 2003.
Zelinski et al., Cancer Res., 61:2301, 2001.
Zhang et al., J. Biol. Chem., 279:10677-684, 2004.
Zhou and Giannakakou, Curr. Med. Chem. Anticancer Agents, 5:65-71, 2005.

1. An isolated nucleic acid molecule comprising an inhibitory nucleic acid that hybridizes to a nucleic acid sequence encoding a prefoldin (PFDN) and inhibits the expression of a prefoldin (PFDN) protein.

2. The nucleic acid of claim 1, wherein said prefoldin (PFDN) is PFDN4.

3. The nucleic acid of claim 1, wherein the inhibitory nucleic acid is an siRNA, an sh RNA, a ds RNA, an antisense oligonucleotide, a ribozyme, or a nucleic acid encoding thereof.

4. The nucleic acid of claim 3, wherein the nucleic acid is an siRNA or a nucleic acid encoding an siRNA.

5. The nucleic acid of claim 4, wherein the siRNA is a double stranded nucleic acid of 19 to 100 nucleobases.

6. The nucleic acid of claim 5, wherein the siRNA is 19 to 30 nucleobases.

7. The composition of claim 8, further comprising a chemotherapeutic agent or other anti-cancer agent.

8. A pharmaceutical composition comprising one or more said nucleic acid molecules of claim 1 and a pharmaceutically acceptable carrier.

9. The composition of claim 8, wherein the composition further comprises a lipid component.

10. The composition of claim 9, wherein the lipid component forms a liposome.

11. The composition of claim 9, wherein the lipid component comprises one or more phospholipids.

12. The composition of claim 9, wherein the nucleic acid molecule is encapsulated in the lipid component.

13. The composition of claim 9, wherein lipid component comprises a neutral lipid.

14. The composition of claim 9, wherein the lipid component comprises a positively charged lipid or a negatively charged lipid.

15. A method of treating a cancer or a hyperplastic condition in a subject comprising administering an effective amount of a composition in accordance with claim 8.

16. The method of claim 15, wherein the subject is a human subject.

17. The method of claim 15, wherein the cancer is a cancer in ovary, bladder, blood, bone, bone marrow, brain, breast, colon, esophagus, gastrointestine, gum, head, kidney, liver, lung, nasopharynx, neck, prostate, skin, stomach, testis, tongue, or uterus.

18. The method of claim 17, wherein the cancer is ovarian cancer.

19. The method of claim 15, further comprising administering an additional therapy to the subject.

20. The method of claim 19, wherein the additional therapy comprises administering a chemotherapeutic, a surgery, a radiation therapy or a gene therapy.

21. The method of claim 20, wherein the additional therapy comprises administering a chemotherapeutic.

22. The method of claim 21, wherein the chemotherapeutic comprises docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin, methotrexate, or combinations thereof.

23. The method of claim 22, wherein the chemotherapeutic comprise docetaxel or paclitaxel.

24. The nucleic acid of claim 3, wherein the inhibitory nucleic acid is an sh RNA, a ds RNA, an antisense oligonucleotide, a ribozyme, or a nucleic acid encoding thereof.

25. The nucleic acid of claim 3, wherein the inhibitory nucleic acid is a ds RNA.

26. The nucleic acid of claim 3, wherein the inhibitory nucleic acid is a ribozyme.

27. The composition of claim 8, wherein the nucleic acid is a siRNA or a nucleic acid encoding a siRNA.

28. The composition of claim 27, wherein the siRNA is a double stranded nucleic acid of 19 to 100 nucleobases.

29. The composition of claim 28, wherein the siRNA is a double stranded nucleic acid of 19 to 30 nucleobases.

30. The composition of claim 7, wherein the chemotherapeutic agent or other anti-cancer agent is docetaxel, paclitaxel, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin, or methotrexate.

31. The composition of claim 30, wherein the chemotherapeutic agent or other anticancer agent is docetaxel.

32. The composition of claim 31, wherein the chemotherapeutic agent or other anti-cancer agent is paclitaxel.
